(19) United States
(12) Patent Application Publication
(10) Pub. No.: US 2026/0159604 A1
(43) Pub. Date: Jun. 11, 2026

(54) USES OF NON-SMALL CELL LUNG CANCER TARGET ARID1A AND INHIBITOR THEREOF IN PREPARATION OF DRUG FOR TREATING LUNG CANCER

(71) Applicant: THE FIRST AFFILIATED HOSPITAL OF BENGBU MEDICAL COLLEGE, Bengbu, Anhui (CN)

(72) Inventors: Xiaojing WANG, Bengbu, Anhui (CN); Fei LIU, Bengbu, Anhui (CN); Linling ZHANG, Bengbu, Anhui (CN); Chao MA, Bengbu, Anhui (CN); Hangtian ZHOU, Bengbu, Anhui (CN); Lingling XU, Bengbu, Anhui (CN); Nan WU, Bengbu, Anhui (CN); Fuliang CHEN, Bengbu, Anhui (CN)

(21) Appl. No.: 18/705,579

(22) PCT Filed: Oct. 27, 2022
(86) PCT No.: PCT/CN2022/128046

§ 371 Date: Apr. 28, 2024

(30) Foreign Application Priority Data
Oct. 27, 2021 (CN) 202111256243.8

(51) Int. Cl. C07K 16/30; A61K 31/7088; A61K 38/00; A61K 48/00; A61P 35/00; G01N 33/5752

(52) U.S. Cl. CPC C07K 16/3023; A61K 31/7088; A61K 38/00; A61K 48/00; A61P 35/00; G01N 33/5752; G01N 2800/52; G01N 2800/7028

(57) ABSTRACT

A non-small cell lung cancer target ARID1A and an inhibitor thereof can be used in the preparation of a drug for treating lung cancer. A glycolysis inhibitor is used for preparing a composition or a preparation. The composition or the preparation is used for: (a) preventing and/or treating lung cancer, wherein the lung cancer is an ARID1A-negative lung cancer, and/or (b) inhibiting lung cancer cells, wherein the lung cancer cells are ARID1A-negative lung cancer cells.

### INCORPORATION OF SEQUENCE LISTING

This application contains a sequence listing submitted in Computer Readable Form (CRF). The CRF file contains the sequence listing entitled “PBA4080165-SequenceListing.xml”, which was created on Apr. 26, 2024, and is 13,953 bytes in size. The information in the sequence listing is incorporated herein by reference in its entirety.

### TECHNICAL FIELD

The present invention relates to the field of biopharmaceutical technology, specifically to the uses of a non-small cell lung cancer target ARID1A and an inhibitor thereof in the preparation of a drug for treating lung cancer.

### BACKGROUND

The incidence rate and mortality of lung cancer rank first among malignant tumors in China and around the world, posing a great threat to people's lives and health. However, the pathogenesis of lung cancer has not yet been fully clarified, which has seriously hindered the progress of lung cancer prevention and treatment.

Through in-depth researches on the molecular events involved in the genesis and development of lung cancer, it is particularly important to identify new diagnostic biomarkers and intervention targets thereof for improving the overall survival rate of lung cancer.

The activation of oncogenes/inactivation of anti-oncogenes is the fundamental cause of cancer genesis. On the one hand, cancer genesis is regulated by genomic variations; and on the other hand, it is influenced by epigenetic modifications. Chromatin remodeling is an important way of epigenetic modifications. ARID1A is an important subunit of chromatin remodeling complex SWI/SNF, which participates in regulating the expression of various cancer-related genes and plays an important role in the genesis and development of various malignant tumors. However, its role in the pathogenesis of non-small cell lung cancer has not yet been reported.

Given the diverse pathogenesis of lung cancer, there is an urgent need in this field to develop new targets for lung cancer treatment and corresponding treatment methods for these new targets, thereby effectively preventing and treating lung cancer.

### SUMMARY OF THE INVENTION

The purpose of the present invention is to provide new targets for lung cancer treatment and corresponding treatment methods for these new targets.

In the first aspect of the present invention, it provides a use of a glycolysis inhibitor in the manufacture of a composition or preparation, wherein the composition or preparation is used for: (a) preventing and/or treating lung cancer, wherein the lung cancer is an ARID1A-negative lung cancer; and/or (b) inhibiting lung cancer cells, wherein the lung cancer cells are ARID1A-negative lung cancer cells.

In another preferred embodiment, the lung cancer is a lung cancer of mammals (including human and non-human mammals).

In another preferred embodiment, the ARID1A-negative refers to a significant decrease in ARID1A expression and/or activity compared to normal control cells.

In another preferred embodiment, the significant decrease refers that the ratio of ARID1A expression A1 in lung cancer cells or tissues to ARID1A expression E0 in normal lung cells or tissues (i.e., E1/E0) is ≤½, preferably ≤⅓, more preferably ≤⅕; and/or the ratio of ARID1A activity A1 in lung cancer cells or tissues to ARID1A activity A0 in normal lung cells or tissues (i.e., A1/A0) is ≤½, preferably ≤⅓, more preferably ≤⅕.

In another preferred embodiment, the expression of BAF250 or a subunit thereof is downregulated, or the activity of BAF250 or a subunit thereof is significantly decreased in the lung cancer or lung cancer cells.

In another preferred embodiment, the level of SWI/SNF complex is downregulated, or the activity of SWI/SNF complex is significantly decreased in the lung cancer or lung cancer cells.

In another preferred embodiment, the lung cancer is an ARID1A-deficient lung cancer.

In another preferred embodiment, the lung cancer is selected from the group consisting of: adenocarcinoma, squamous cell carcinoma, and a combination thereof.

In another preferred embodiment, the lung cancer is selected from the group consisting of: small cell lung cancer and non-small cell lung cancer.

In another preferred embodiment, the glycolysis inhibitor is selected from the group consisting of:
(Z1) a Hif-la inhibitor;
(Z2) a Pgk1 inhibitor;
(Z3) a Pgam1 inhibitor;
(Z4) a Pkm2 inhibitor;
(Z5) any combination of Z1 to Z4 mentioned above.

In another preferred embodiment, the glycolysis inhibitor inhibits Pgk1, Pgam1, and/or Pkm2.

In another preferred embodiment, the glycolysis inhibitor is selected from the group consisting of: small molecule compound, antibody, antisense nucleic acid, gene editing drug, and a combination thereof.

In another preferred embodiment, the inhibitor is selected from a small molecule compound, antibody, siRNA, shRNA, or CRISPR/Cas editing tool-mediated inhibitory complex.

In another preferred embodiment, the inhibitor comprises a Pgk1-inhibiting antisense nucleic acid, a Pgaml-inhibiting antisense nucleic acid, and/or a Pkm2-inhibiting antisense nucleic acid.

In another preferred embodiment, the inhibitor for inhibiting Pgam1 has the sequence shown in SEQ ID NO: 4: GGATTGCTCTCTTCTGCACAG; In another preferred embodiment, the inhibitor for inhibiting Pgam1 has the sequence shown in SEQ ID NO: 5: TTGACCAGATGTGGTTGCCAG.

In another preferred embodiment, the inhibitor for inhibiting Pkm2 has the sequence shown in SEQ ID NO: 6: GGGGCAGAGTCAATGTCCAGG; In another preferred embodiment, the inhibitor for inhibiting Pgam1 has the sequence shown in SEQ ID NO: 7: CCGAAGCCACACAGTGAAGCA.

In another preferred embodiment, the glycolysis inhibitor is selected from the group consisting of: a BETi small molecule compound, a 2-DG small molecule (2-deoxy-D-glucose, FIG. 1 panel a), and a combination thereof.

In another preferred embodiment, the BETi small molecule compound is selected from the group consisting of: JQ1 (FIG. 1 panel b), BET762 (Molibresib, FIG. 1 panel c), OXT015 (Birabresib, FIG. 1d), BET726 (FIG. 1 panel e), BET151 (FIG. 1 panel f), and a combination thereof.

In another preferred embodiment, the gene editing drug is used to inhibit or eliminate the expression of Pgk1 gene, Pgam1 gene, and/or Pkm2 gene.

In another preferred embodiment, the treatment comprises: inhibiting lung cancer cell proliferation rate, altering lung cancer cell cycle distribution, promoting lung cancer cell apoptosis, inhibiting lung cancer tissue growth, and a combination thereof.

In the second aspect of the present invention, it further provides a use of an ARID1A gene, mRNA, cDNA, protein, or a detection reagent thereof in the manufacture of a kit, wherein the kit is used for one or more uses selected from the group consisting of:
(i) evaluating whether a lung cancer patient is suitable for treatment with a glycolysis inhibitor; and/or
(ii) evaluating the prognosis of a lung cancer patient treated with a glycolysis inhibitor.

In another preferred embodiment, the kit is also used for:
(iii) detection of lung cancer or the risk of lung cancer;
(iv) prognostic assessment of a lung cancer patient.

In another preferred embodiment, the kit is used for classifying a lung cancer patient based on ARID1A expression and/or activity.

In another preferred embodiment, the detection reagent comprises:
(a) an ARID1A-specific antibody, an ARID1A-specific binding molecule; and/or
(b) a primer or a primer pair, a probe, or a chip for specific amplification of ARID1A mRNA or ARID1A cDNA.

In the third aspect of the present invention, it further provides a product combination, wherein the product comprises:
(a) a detection reagent or a kit containing the detection reagent, wherein the detection reagent is a detection reagent for detecting ARID1A gene, mRNA, cDNA, protein, and a combination thereof; and
(b) a pharmaceutical composition, wherein the pharmaceutical composition comprises a glycolysis inhibitor as an active ingredient and a pharmaceutically acceptable carrier.

In another preferred embodiment, the kit comprises a container, wherein the container comprises a detection reagent for detecting ARID1A gene, mRNA, cDNA, protein, and a combination thereof; and a label or instructions indicating that the kit is used to evaluate whether a lung cancer patient is suitable for treatment with a glycolysis inhibitor.

In another preferred embodiment, the kit further comprises ARID1A gene, mRNA, cDNA, and/or protein as a reference substance or quality control substance.

In the fourth aspect of the present invention, it further provides a use of the product combination of the third aspect of the present invention in the manufacture of a medical product for treating ARID1A-negative lung cancer.

In the fifth aspect of the present invention, it further provides a method for treating lung cancer, which comprises the steps of:
(a) classifying a lung cancer patient based on ARID1A expression and/or activity, thereby classifying the patient into an ARID1A-positive lung cancer patient or an ARID1A-negative lung cancer patient; and
(b) administering a glycolysis inhibitor to the ARID1A-negative lung cancer patient.

In another preferred embodiment, in step (b), it further comprises: detecting ARID1A expression and/or activity during the treatment process.

In another preferred embodiment, the glycolysis inhibitor is administered to humans.

It should be understood that within the scope of the present invention, the above-mentioned technical features of the present invention and the technical features specifically described in the following (such as the examples) can be combined with each other to form new or preferred technical solutions, which are not redundantly repeated one by one due to space limitation.

### DESCRIPTION OF THE DRAWINGS

FIG. 1 shows the structural diagram of small molecule compounds that intervene in glucose metabolism levels and key genes involved in glycolysis.

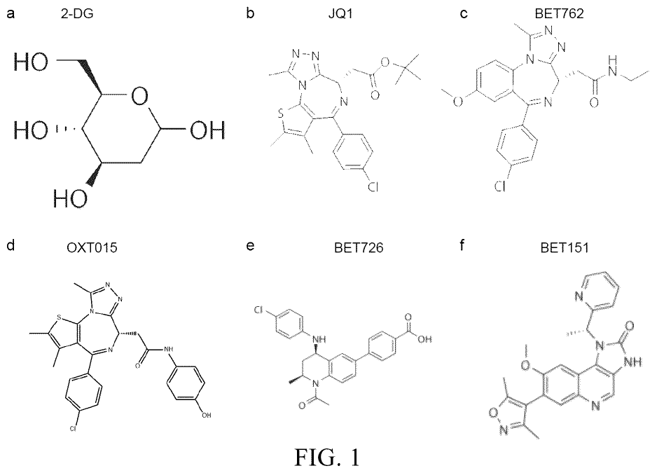

FIG. 2 shows that the ARID1A gene had a mutation frequency of −10% in the cohort of lung cancer patients, with missense mutations and truncating mutations being the main mutations.

FIG. 3 shows that the inactivation of ARID1A gene could significantly promote the tumorigenic ability of mouse lung tissues under the background of KrasG12D overexpression and Tp53 deletion.

FIG. 4 shows that the ARID1A gene mutations significantly activated the glycolysis signaling pathway in tumor cells. The expressions of key genes Pgam1, Pkm and Pgk1 in glycolysis were significantly upregulated.

FIG. 5 shows that the ARID1A gene mutations led to the remodeling of chromatin state and opened up the promoter regions of glycolysis-associated genes Pgam1, Pkm and Pgk1. Among them, the key gene Hif-la of hypoxia pathway could bind more tightly to the open promoter regions of key genes in glycolysis in the absence of ARID1A, thereby promoting the expression levels thereof.

FIG. 6 suggests that the progression of ARID1A-deficient lung cancers could be inhibited by directly inhibiting the glycolysis levels of tumor cells or downregulating the expression levels of key genes in glycolysis.

FIG. 7 suggests that inhibiting the SWI/SNF complex through BETi small molecules could slow down the progression of ARID1A-deficient lung cancers, wherein JQ1 had a more prominent inhibitory effect.

### DETAILED DESCRIPTION

After extensive and in-depth researches, it has been found in the present application that ARID1A can act as an anti-oncogene to regulate tumors, and ARID1A mutations can promote the glycolysis levels of tumor cells. ARID1A provides a new target for the treatment of ARID1A-deficient lung cancers. One or more inhibitors thereof can inhibit lung cancer cell proliferation rate, alter lung cancer cell cycle distribution, promote lung cancer cell apoptosis, or inhibit lung cancer tissue growth, thereby treating lung cancers, and opening up new directions for lung cancer treatment.

### Terms

In order to make it easier to understand the present disclosure, certain terms are first defined. As used herein, each of the following terms shall have the meanings given below unless expressly provided herein.

The term “about” may refer to a value or composition within an acceptable error range of a particular value or composition determined by those of ordinary skill in the art, which will depend in part on how the value or composition is measured or determined.

The term “administration” refers to the physical introduction of the product of the present invention into a subject using any one of various methods and delivery systems known to those skilled in the art, including intravenous, intratumoral, intramuscular, subcutaneous, intraperitoneal, spinal or other parenteral routes of administration, such as by injection or infusion.

The terms “p53” and “Tp53” can be used interchangeably to refer to a tumor suppressor gene. The protein encoded by this gene is a transcription factor that controls the initiation of the cell cycle. The p53 gene plays a role of slowing or monitoring cell division under normal circumstances.

### ARID1A Gene

Specifically, in the present invention, the amino acid sequence of the human ARID1A is as shown in SEQ ID NO: 1, and the amino acid sequence of the mouse ARID1A is as shown in SEQ ID NO: 2. Inactivation refers to the mutation of the nucleic acid encoding the ARID1A protein, resulting in changes in the sequence and structure of the encoded amino acid sequence thereof, leading to the loss of biological function of the ARID1A protein.

In another preferred embodiment, the mutation of the nucleic acid refers to a replacement of the base sequence;

In another preferred embodiment, the mutation of the nucleic acid refers to a base deletion;

In another preferred embodiment, the mutation of the nucleic acid refers to a base sequence insertion.

In another preferred embodiment, the mutation of the nucleic acid refers to fusion between bases; In another preferred embodiment, the mutation of the nucleic acid refers to an abnormal base amplification. The mutation of the nucleic acid ultimately leads to the formation of a missense mutation or a nonsense mutation.

Especially, no high-frequency hot spot mutations are found in ARID1A mutations in lung cancers, and the mutation sites thereof cover the entire coding region.

In another preferred embodiment, the mutation sites exist in the DNA binding domain thereof;

In another preferred embodiment, the variable sites are located at the BAF250 subunit, i.e., BAF250a of the SWI/SNF complex, or other undefined structural regions.

Especially, through detailed biological data analysis of the present invention, the mutation frequency of ARID1A in non-small cell lung cancers is approximately-11%, and ARID1A belongs to a significantly high-frequency mutation gene and has significant research values.

Specifically, deletion mutations in the ARID1A sequence can promote the progression of lung adenocarcinomas.

Firstly, the research objects of the present invention are KrasLSL-G12D; P53Flox/Flox mouse animal models (KP mice), which can effectively simulate the physiological characteristics of human lung cancers. Overexpression of KrasG12D mutants in mouse lung cells and simultaneous knockout of the P53 anti-oncogene can be induced by introducing a gene fragment (SEQ ID NO: 3) containing the Cre recombinase via an AAV virus vector. This method is a well-known method for establishing a lung adenocarcinoma model in this field.

Secondly, based on the KP mouse models, an ARID1A conditional knockout mouse system (KrasLSL-G12D; P53Flox/Flox; ARID1A Flox/Flox, KPA) is constructed. Overexpression of KrasG12D mutants in mouse lung cells and simultaneous knockout of the P53/ARID1A anti-oncogene can be induced under the expression of Cre recombinase. This embodiment can effectively study the function of ARID1A gene in lung adenocarcinomas.

Finally, the present invention suggests that the loss of ARID1A gene function can significantly promote the proliferation and invasion ability of lung adenocarcinoma cells, and significantly reduce the survival period of mice.

### Mechanism of ARID1A Gene Deletion Promoting Disease Progression

The mechanism of ARID1A gene deletion promoting the progression of lung adenocarcinoma diseases is described as follows:
1) ARID1A inactivating mutations cause chromatin remodeling in tumor cells, altering the chromatin opening state;
2) The chromatin opening includes an increased openness of the promoter regions of genes related to glycolysis pathways, and related genes include but are not limited to Pgk1, Pgaml, and Pkm2.
3) ARID1A inactivating mutations promote a more significant binding of HIF-la, a key gene in the hypoxia signaling pathway, to the promoter regions of genes such as Pgk1, Pgaml, and Pkm2, wherein the binding can significantly promote the expression levels of these genes.

Specifically, an increase in glycolysis level can promote tumor cell progression, and this result is close to the theoretical knowledge well-known in this field. During the verification process of the present invention, transcriptome sequencing, CHIP-SEQ sequencing, RT-PCR validation, and bioinformatics analysis are widely used for verification. The data collected by the present invention are based on a large number of clinical samples and related model animal studies.

In another preferred embodiment, reducing glycolysis level can inhibit the progression of ARID1A-mutant lung adenocarcinomas.

In another preferred embodiment, reducing glycolysis level can be achieved through small molecule inhibitors, such as the well-known 2-DG drug in this field.

In another preferred embodiment, reducing glycolysis level can be achieved through traditional techniques such as siRNA, shRNA, antibodies, etc., to inhibit the expressions of Pgk1, Pgam1, and Pkm2 genes.

In another preferred embodiment, reducing glycolysis level can be achieved through gene editing, such as CRISPR/Cas related techniques, to inhibit the expressions of Pgk1, Pgam1, and Pkm2 genes.

Specifically, the methods of reducing glycolysis level as described above can be used to prepare lung cancer treatment drugs.

The drugs can be a single interference method or any combination of the above methods, and can also be a combination medication consisting of any one of the above methods and a well-known clinical drug.

The forms of the lung cancer treatment drugs have no special restrictions, and can be various substance forms such as solid, liquid, gel, semifluid, aerosol, etc.

The lung cancer treatment drugs mainly target mammals, such as rodents, primates, etc.

In another preferred embodiment, the drugs can inhibit the cell proliferation rate and alter the cell cycle distribution, etc., of ARID1A-deficient non-adenocarcinoma cells.

### Gene BETi

One or more Bromodomain and extra terminal protein (BET) inhibitor (BETi) small molecule compounds described in the present invention can effectively inhibit the proliferation of ARID1A-mutant lung cancer cells, improve the survival period, and provide a novel method for inhibiting the progression of ARID1A-mutant lung cancers.

BETi small molecule compounds include but are not limited to JQ1, BET762, OXT015, BET726, and BET151, etc. Among them, ARID1A-deficient lung adenocarcinoma cells are more sensitive to JQ1 and BET762. Reported studies have shown that molecules such as JQ1 also have inhibitory effects on ARID1A-mutant ovarian cancers. The present invention supports and expands the intervention effects of BETi on ARID1A-deficient cancer tissues. Specifically, the researches related to the present invention are based on KP and KPA mouse model animals, as well as organoids and transplanted tumor models, etc., constructed on the basis of mouse cells.

In another preferred embodiment, the present invention suggests that BETi has strong inhibitory effects on the activity, proliferation ability, and in vivo tumor burden of ARID1A-deficient lung cancer cells.

Effective doses of BETi inhibitors and effective doses of at least one of other lung cancer treatment drugs can be administered synchronously or sequentially.

The gene BETi is a first discovered therapeutic target for ARID1A-deficient lung cancers in the present invention. When used in combination with other lung cancer treatment drugs other than this inhibitor, at least a combined therapeutic effect can be achieved, thereby further enhancing the therapeutic effect on lung cancers.

Other lung cancer treatment drugs include but are not limited to antibody drugs, chemical drugs, or targeted drugs, etc.

In another preferred embodiment, the BETi small molecule can inhibit the cell proliferation rate and alter the cell cycle distribution, etc., of ARID1A-deficient non-adenocarcinoma cells.

### Lung Cancer

Lung cancers occur in the epithelium of bronchial mucosa, also known as bronchial lung cancers, which generally refer to the cancers of the lung parenchyma. Lung cancers are one of the malignant tumors with the fastest growth in incidence rate and mortality and with the greatest threat to human health and lives. Lung cancers can grow into the lumen of the bronchi or/and adjacent lung tissues, and can spread through lymph node metastasis, blood metastasis or bronchial metastasis.

Lung adenocarcinomas are a type of lung cancers that belong to non-small cell carcinomas. Unlike squamous cell lung cancers, lung adenocarcinomas are more likely to occur in women and non-smokers. Lung adenocarcinomas originate from the epithelium of bronchial mucosa, with a few originating from the mucous glands of the major bronchi. Compared with squamous cell carcinomas and undifferentiated carcinomas, lung adenocarcinomas have a lower incidence rate, a younger onset age, and are more common in women. Most adenocarcinomas originate from smaller bronchus and are peripheral lung cancers.

Non-small-cell lung carcinomas account for approximately 80% of all lung cancers, including squamous cell carcinomas (squamous carcinomas), adenocarcinomas, and large-cell carcinomas. Compared with small-cell carcinomas, cancer cells of non-small-cell lung carcinomas grow and divide slower, and the spread and metastasis thereof are relatively late. About 75% of patients with non-small cell lung carcinomas are already in the middle and late stages at the time of discovery, with a very low 5-year survival rate.

### Chromatin Remodeling

Chromatin remodeling is a dynamic process in which the phase of nucleosomes is adjusted to neutralize the positive charge of basic amino acid residues (lysine K, arginine R, histidine H, etc.) in histone tails, weaken the binding of basic amino acids to DNA in nucleosomes, reduce the aggregation between adjacent nucleosomes, and allow nucleosomes to slide and expose previously masked elements, or instantly expose elements on the surface of nucleosomes.

Chromatin remodeling is the main regulatory mode that controls gene expression at the epigenetic level, including: ATP-dependent chromatin physical modifications and covalent chemical modifications of chromatin. Among them, ATP-dependent chromatin physical modifications, namely ATP hydrolysis, enable nucleosomes to slide along DNA, or cause nucleosomes to dissociate and reassemble. Due to the presence of nucleosomes around RNA polymerase II during elongation, and theses nucleosomes are in a dynamic equilibrium state of partial dissociation and partial assembly, these chromatin physical remodeling complexes are also of great significance for transcriptional elongation.

ATP-dependent chromatin physical modifications are achieved through the action of ATP-dependent chromatin remodeling complexes. Most of these remodeling complexes are multi-protein subunit complexes with ATP hydrolases as the catalytic center. According to the different sequences and structures of ATP hydrolases, the remodeling complexes can be divided into at least five categories: SWI/SNF family complexes, ISWI family complexes, CHD family complexes, INO80 family complexes, and SWR1.

Among the different chromatin physical modifications of remodeling complexes, SWI/SNF remodeling complexes mainly disrupt the order of nucleosomes. INO80 and SWI/SNF family complexes are involved in DNA double strand break (DSB) repair and nucleotide excision repair (NER). Therefore, remodeling complexes play a central role in the p53-mediated response to DNA damage.

Various structural domains in ATP-dependent chromatin remodeling complexes play a role in nucleosome recognition: Bromodomain is a common motif, approximately 110 aa, connected by ring regions with variable length in 4 α-helices to form a hydrophobic pocket that can recognize acetylated lysine residues; and chomodomain (CHD) often appears in two tandem structures at the N-terminus, capable of binding to methylated lysine residues.

### Glycolysis Pathway

The glycolysis pathway, also known as the EMP pathway, is a series of reactions that degrade glucose and glycogen into pyruvate, accompanied by ATP production. It is a common pathway for glucose degradation in all living organisms. The glycolysis pathway can occur under both anaerobic and aerobic conditions, and is a common metabolic pathway for aerobic or anaerobic decomposition of glucose. Tumor cells are in a state of uncontrolled division and proliferation, with a particularly strong demand for energies. However, the process of energy production by tumor cells does not mainly rely on classical mitochondrial oxidative phosphorylation. On the contrary, cancer cells and other proliferating cells often choose the glycolysis pathway to obtain the energies they need, even in the presence of sufficient oxygen, and this is well-known as the “Warburg Effect”.

Phosphoglycerate kinase 1 (PGK1) is a key enzyme in the glycolysis process, catalyzing 1,3-diphosphoglycerate to produce 3-phosphoglycerate and simultaneously producing ATP, thus playing an important role in cellular energy metabolism. The severity of liver cancer patients is positively correlated with the expression level of PGK1 protein. After knocking down the Pgk1 gene, the glycolysis ability and production capacity of liver cancer cell lines decreased, cell proliferation was inhibited, and tumor formation ability was weakened.

Phosphoglycerate mutase 1 (PGAM1) is one of the important functional enzymes in the glycolysis pathway, catalyzing the conversion of 3-phosphoglycerate (3-PG) to 2-phosphoglycerate (2-PG), promoting glucose metabolism and energy generation. It affects other metabolic pathways by regulating the conversion balance between 3-PG and 2-PG, participates in the synthesis of intracellular biomacromolecules and maintenance of redox homeostasis, promoting the proliferation and metastasis of tumor cells. PGAM1 is generally highly expressed in various malignant tumors, including non-small cell lung cancers, and is positively correlated with poor prognosis.

Pyruvate kinase M2 isozyme (PKM2) is a key enzyme in the final step of aerobic glycolysis process in tumor cells, which breaks down phosphoenolpyruvate (PEP) into pyruvate, enabling various cancer cells to obtain energies. Researches have shown that PKM2 not only plays an important role in tumor cell metabolism, but also serves as an important signaling molecule in tumor cell proliferation, transformation, and prognosis.

p53/Tp53

P53/TP53 is a tumor suppressor gene, and mutations in this gene occur in over 50% of all malignant tumors. The protein encoded by this gene is a transcription factor that controls the initiation of the cell cycle. Many signals related to cellular health are sent to the p53 protein. Whether or not cell division begins is determined by this protein. If the cell is damaged and cannot be repaired, the p53 protein will participate in the initiation process, causing the cell to die during apoptosis. P53-deficient cells do not have this control and even continue to divide under unfavorable conditions. Like all other tumor suppressors, the p53 gene plays a role in slowing or monitoring cell division under normal circumstances. After a mutation in the p53 gene, due to a change in its spatial conformation, if it loses its regulatory function in cell growth, apoptosis, and DNA repair, the p53 gene will transform from an anti-oncogene to an oncogene.

### Companion Diagnostic

Companion diagnostic is a type of in vitro diagnosis that provides crucial information for the safe and effective use of corresponding therapeutic products (drugs or biological products). In order to use the corresponding therapeutic products safely and effectively, companion diagnostic tests are essential, and the purposes thereof include: (1) identifying patients who are likely to benefit from the therapeutic products; (2) identifying patients who may increase the risk of serious adverse reactions due to the use of the therapeutic products for treatment; (3) monitoring the response of the therapeutic products to treatment, thereby adjusting treatment (such as treatment plan, dosage, withdrawal) to better achieve safety and effectiveness, etc. Companion diagnostic includes reagents used for specific tests, quality control samples, and complete equipment, etc., and is a complete set of detection system.

The main advantages of the present invention
(a) The present invention elucidates the role of ARID1A gene deletion or ARID1A functional inactivation in promoting lung cancer progression, and proposes a regulatory pathway dependent on hypoxia and glycolysis signaling pathways.
(b) The present invention proposes a targeted treatment method for ARID1A-inactivated lung cancers based on key genes of hypoxia and glycolysis signaling pathways. These methods include, but are not limited to, and can be selected from small molecule compounds, antibodies, siRNA, shRNA, or CRISPR/Cas editing tool-mediated inhibitory complexes. The present invention provides a new and effective treatment method for lung cancers.

The present invention is further explained below in conjunction with specific examples. It should be understood that these examples are only for illustrating the present invention and not intend to limit the scope of the present invention. The experimental method without detailed conditions specified in the following embodiments is generally in accordance with conventional conditions, such as those described in Sambrook et al., Molecular Cloning: A Laboratory Manual (New York: Cold Spring Harbor Laboratory Press, 1989), or in accordance with the conditions recommended by the manufacturers. Unless otherwise stated, percentages and parts are calculated by weight.

Amino acid sequence and nucleotide sequence
SEQ ID NO: 1
MAAQVAPAAASSLGNPPPPPPSELKKAEQQQREEAGGEAAAAAAAERGEMKAAAGQ
ESEGPAVGPPQPLGKELQDGAESNGGGGGGGAGSGGGPGAEPDLKNSNGNAGPRPALNNN
LTEPPGGGGGGSSDGVGAPPHSAAAALPPPAYGFGQPYGRSPSAVAAAAAAVFHQQHGGQ
QSPGLAALQSGGGGGLEPYAGPQQNSHDHGFPNHQYNSYYPNRSAYPPPAPAYALSSPRGG
TPGSGAAAAAGSKPPPSSSASASSSSSSFAQQRFGAMGGGGPSAAGGGTPQPTATPTLNQLL
TSPSSARGYQGYPGGDYSGGPQDGGAGKGPADMASQCWGAAAAAAAAAAASGGAQQRS
HHAPMSPGSSGGGGQPLARTPQPSSPMDQMGKMRPQPYGGTNPYSQQQGPPSGPQQGHGY
PGQPYGSQTPQRYPMTMQGRAQSAMGGLSYTQQIPPYGQQGPSGYGQQGQTPYYNQQSPH
PQQQQPPYSQQPPSQTPHAQPSYQQQPQSQPPQLQSSQPPYSQQPSQPPHQQSPAPYPSQQST
TQQHPQSQPPYSQPQAQSPYQQQQPQQPAPSTLSQQAAYPQPQSQQSQQTAYSQQRFPPPQ
ELSQDSFGSQASSAPSMTSSKGGQEDMNLSLQSRPSSLPDLSGSIDDLPMGTEGALSPGVSTS
GISSSQGEQSNPAQSPFSPHTSPHLPGIRGPSPSPVGSPASVAQSRSGPLSPAAVPGNQMPPRP
PSGQSDSIMHPSMNQSSIAQDRGYMQRNPQMPQYSSPQPGSALSPRQPSGGQIHTGMGSYQ
QNSMGSYGPQGGQYGPQGGYPRQPNYNALPNANYPSAGMAGGINPMGAGGQMHGQPGIP
PYGTLPPGRMSHASMGNRPYGPNMANMPPQVGSGMCPPPGGMNRKTQETAVAMHVAAN
SIQNRPPGYPNMNQGGMMGTGPPYGQGINSMAGMINPQGPPYSMGGTMANNSAGMAASP
EMMGLGDVKLTPATKMNNKADGTPKTESKSKKSSSSTTTNEKITKLYELGGEPERKMWVD
RYLAFTEEKAMGMTNLPAVGRKPLDLYRLYVSVKEIGGLTQVNKNKKWRELATNLNVGT
SSSAASSLKKQYIQCLYAFECKIERGEDPPPDIFAAADSKKSQPKIQPPSPAGSGSMQGPQTP
QSTSSSMAEGGDLKPPTPASTPHSQIPPLPGMSRSNSVGIQDAFNDGSDSTFQKRNSMTPNPG
YQPSMNTSDMMGRMSYEPNKDPYGSMRKAPGSDPFMSSGQGPNGGMGDPYSRAAGPGLG
NVAMGPRQHYPYGGPYDRVRTEPGIGPEGNMSTGAPQPNLMPSNPDSGMYSPSRYPPQQQ
QQQQQRHDSYGNQFSTQGTPSGSPFPSQQTTMYQQQQQNYKRPMDGTYGPPAKRHEGEM
YSVPYSTGQGQPQQQQLPPAQPQPASQQQAAQPSPQQDVYNQYGNAYPATATAATERRPA
GGPQNQFPFQFGRDRVSAPPGTNAQQNMPPQMMGGPIQASAEVAQQGTMWQGRNDMTY
NYANRQSTGSAPQGPAYHGVNRTDEMLHTDQRANHEGSWPSHGTRQPPYGPSAPVPPMTR
PPPSNYQPPPSMQNHIPQVSSPAPLPRPMENRTSPSKSPFLHSGMKMQKAGPPVPASHIAPAP
VQPPMIRRDITFPPGSVEATQPVLKQRRRLTMKDIGTPEAWRVMMSLKSGLLAESTWALDT
INILLYDDNSIMTFNLSQLPGLLELLVEYFRRCLIEIFGILKEYEVGDPGQRTLLDPGRFSKVSS
PAPMEGGEEEEELLGPKLEEEEEEEVVENDEEIAFSGKDKPASENSEEKLISKFDKLPVKIVQ
KNDPFVVDCSDKLGRVQEFDSGLLHWRIGGGDTTEHIQTHFESKTELLPSRPHAPCPPAPRK
HVTTAEGTPGTTDQEGPPPDGPPEKRITATMDDMLSTRSSTLTEDGAKSSEAIKESSKFPFGIS
PAQSHRNIKILEDEPHSKDETPLCTLLDWQDSLAKRCVCVSNTIRSLSFVPGNDFEMSKHPG
LLLILGKLILLHHKHPERKQAPLTYEKEEEQDQGVSCNKVEWWWDCLEMLRENTLVTLANI
SGQLDLSPYPESICLPVLDGLLHWAVCPSAEAQDPFSTLGPNAVLSPQRLVLETLSKLSIQDN
NVDLILATPPFSRLEKLYSTMVRFLSDRKNPVCREMAVVLLANLAQGDSLAARAIAVQKGS
IGNLLGFLEDSLAATQFQQSQASLLHMQNPPFEPTSVDMMRRAARALLALAKVDENHSEFT
LYESRLLDISVSPLMNSLVSQVICDVLFLIGQS
SEQ ID NO: 2
MAAQVAPAAASSLGNPPPPPSELKKAEQQQREEAGGEAAAAAAERGEMKAAAGQES
EGPAVGPPQPLGKELQDGAESNGGGGGGGAGSGGGPGAEPDLKNSNGNAGPRPALNNNLP
EPPGGGGGGGSSSSDGVGAPPHSAAAALPPPAYGFGQAYGRSPSAVAAAAAAVFHQQHGG
QQSPGLAALQSGGGGGLEPYAGPQQNSHDHGFPNHQYNSYYPNRSAYPPPPQAYALSSPRG
GTPGSGAAAAAGSKPPPSSSASASSSSSSFAQQRFGAMGGGGPSAAGGGTPQPTATPTLNQL
LTSPSSARGYQGYPGGDYGGGPQDGGAGKGPADMASQCWGAAAAAAAAAAAVSGGAQQ
RSHHAPMSPGSSGGGGQPLARTPQSSSPMDQMGKMRPQPYGGTNPYSQQQGPPSGPQQGH
GYPGQPYGSQTPQRYPMTMQGRAQSAMGSLSYAQQIPPYGQQGPSAYGQQGQTPYYNQQ
SPHPQQQPPYAQQPPSQTPHAQPSYQQQPQTQQPQLQSSQPPYSQQPSQPPHQQSPTPYPSQ
QSTTQQHPQSQPPYSQPQAQSPYQQQQPQQPASSSLSQQAAYPQPQPQQSQQTAYSQQRFPP
PQELSQDSFGSQASSAPSMTSSKGGQEDMNLSLQSRPSSLPDLSGSIDDLPMGTEGALSPGVS
TSGISSSQGEQSNPAQSPFSPHTSPHLPGIRGPSPSPVGSPASVAQSRSGPLSPAAVPGNQMPP
RPPSGQSDSIMHPSMNQSSIAQDRGYMQRNPQMPQYTSPQPGSALSPRQPSGGQMHSGVGS
YQQNSMGSYGPQGSQYGPQGGYPRQPNYNALPNANYPNAGMAGSMNPMGAGGQMHGQ
PGIPPYGTLPPGRMAHASMGNRPYGPNMANMPPQVGSGMCPPPGGMNRKTQESAVAMHV
AANSIQNRPPGYPNMNQGGMMGTGPPYGQGINSMAGMINPQGPPYPMGGTMANNSAGM
AASPEMMGLGDVKLTPATKMNNKADGTPKTESKSKKSSSSTTTNEKITKLYELGGEPERKM
WVDRYLAFTEEKAMGMTNLPAVGRKPLDLYRLYVSVKEIGGLTQVNKNKKWRELATNLN
VGTSSSAASSLKKQYIQCLYAFECKIERGEDPPPDIFAAADSKKSQPKIQPPSPAGSGSMQGP
QTPQSTSSSMAEGGDLKPPTPASTPHSQIPPLPGMSRSNSVGIQDAFPDGSDPTFQKRNSMTP
NPGYQPSMNTSDMMGRMSYEPNKDPYGSMRKAPGSDPFMSSGQGPNGGMGDPYSRAAGP
GLGSVAMGPRQHYPYGGPYDRVRTEPGIGPEGNMGTGAPQPNLMPSTPDSGMYSPSRYPP
QQQQQQQQQHDSYGNQFSTQGTPSSSPFPSQQTTMYQQQQQNYKRPMDGTYGPPAKRHE
GEMYSVPYSAGQGQPQQQQLPAAQSQPASQPQAAQPSPQQDVYNQYSNAYPASATAATD
RRPAGGPQNQFPFQFGRDRVSAPPGSSAQQNMPPQMMGGPIQASAEVAQQGTMWQGRND
MTYNYANRQNTGSATQGPAYHGVNRTDEMLHTDQRANHEGPWPSHGTRQPPYGPSAPVP
PMTRPPPSNYQPPPSMPNHIPQVSSPAPLPRPMENRTSPSKSPFLHSGMKMQKAGPPVPASHI
APTPVQPPMIRRDITFPPGSVEATQPVLKQRRRLTMKDIGTPEAWRVMMSLKSGLLAESTW
ALDTINILLYDDNSIMTFNLSQLPGLLELLVEYFRRCLIEIFGILKEYEVGDPGQRTLLDPGRF
TKVYSPAHTEEEEEEHLDPKLEEEEEEGVGNDEEMAFLGKDKPSSENNEEKLVSKFDKLPV
KIVQRNDPFVVDCSDKLGRVQEFDSGLLHWRIGGGDTTEHIQTHFESKIELLPSRPYVPCPTP
PRKHLTTVEGTPGTTEQEGPPPDGLPEKRITATMDDMLSTRSSTLTDEGAKSAEATKESSKF
PFGISPAQSHRNIKILEDEPHSKDETPLCTLLDWQDSLAKRCVCVSNTIRSLSFVPGNDFEMS
KHPGLLLILGKLILLHHKHPERKQAPLTYEKEEEQDQGVSCDKVEWWWDCLEMLRENTLV
TLANISGQLDLSPYPESICLPVLDGLLHWAVCPSAEAQDPFSTLGPNAVLSPQRLVLETLSKL
SIQDNNVDLILATPPFSRLEKLYSTMVRFLSDRKNPVCREMAVVLLANLAQGDSLAARAIA
VQKGSIGNLLGFLEDSLAATQFQQSQASLLHMQNPPFEPTSVDMMRRAARALLALAKVDE
NHSEFTLYESRLLDISVSPLMNSLVSQVICDVLFLIGQS
SEQ ID NO: 3
TCCAATCTCCTGACTGTTCACCAGAACCTCCCTGCGCTGCCAGTAGATGCCACTAG
CGATGAGGTCAGGAAAAATCTCATGGATATGTTTAGGGATAGACAGGCGTTTTCTGAAC
ACACCTGGAAAATGCTGCTTAGCGTGTGCCGATCCTGGGCAGCCTGGTGTAAGCTGAAC
AATCGCAAATGGTTCCCCGCCGAGCCGGAGGACGTGCGCGATTACCTGCTGTATCTCCA
GGCAAGAGGGCTGGCTGTCAAGACTATCCAGCAGCACTTGGGCCAACTGAATATGCTG
CATCGACGCAGCGGGCTCCCCCGGCCTAGCGATTCAAACGCAGTCTCCCTTGTTATGAG
GAGAATTAGAAAGGAAAACGTAGATGCGGGTGAGAGGGCTAAGCAGGCTCTCGCTTTT
GAGCGGACTGATTTCGACCAGGTCAGATCCCTGATGGAGAACAGCGATCGGTGCCAGG
ACATCAGGAACCTCGCATTTCTGGGAATTGCATATAACACACTTCTGCGCATAGCTGAG
ATCGCCCGGATCAGAGTGAAAGACATCAGTCGAACGGACGGCGGCCGGATGCTTATTC
ATATTGGACGCACAAAGACATTGGTCAGCACCGCTGGCGTTGAAAAGGCCTTGTCCCTG
GGCGTAACGAAGCTGGTGGAAAGATGGATCTCAGTGTCCGGCGTGGCTGACGACCCTA
ATAATTACTTGTTCTGTCGAGTGAGAAAAAACGGAGTCGCCGCGCCCTCTGCCACCAGC
CAATTGAGTACACGGGCCCTTGAAGGGATCTTTGAGGCAACCCACCGACTCATATACGG
AGCCAAGGATGACAGTGGCCAGAGGTATCTCGCCTGGTCAGGTCATTCTGCTAGGGTGG
GGGCCGCACGAGACATGGCGCGGGCAGGAGTCTCCATACCAGAGATTATGCAAGCTGG
AGGTTGGACAAATGTGAACATCGTTATGAACTATATCCGCAATCTTGACTCTGAAACCG
GGGCCATGGTGAGACTGCTCGAAGATGGTGAC
SEQ ID NO: 4
GGATTGCTCTCTTCTGCACAG
SEQ ID NO: 5
TTGACCAGATGTGGTTGCCAG
SEQ ID NO: 6
GGGGCAGAGTCAATGTCCAGG
SEQ ID NO: 7
CCGAAGCCACACAGTGAAGCA

### Example 1 Mutations of ARID1A Gene in Lung Cancer Patients

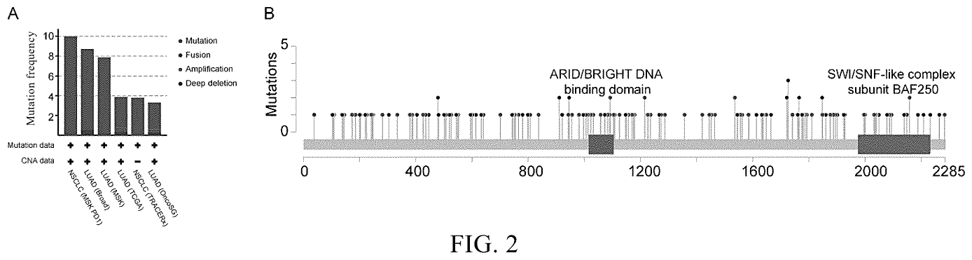

Mutation data from 5 clinical cohorts (including 2,014 lung cancer patients) were analyzed using the cBioPortal tool. Overall, 140 out of 2,014 patients (7%) had mutations in the ARID1A gene, with mutation frequencies ranging from −3.5% to −10%, as shown in FIG. 2 panel A. After analyzing the dataset, we identified 160 mutations in the 140 patients, including 67 missense mutations and 83 other potential inactivating mutations. FIG. 2 panel B represents the mutation status of the ARID1A gene in the clinical cohorts, with green dots indicating missense mutations; black dots indicating truncating mutations. It indicates that the mutations of ARID1A belong to high-frequency mutations in human lung cancers, suggesting that these mutations have important clinical research values.

### Example 2 ARID1A Gene Deletion Promotes the Genesis of In Vivo KrasG12D-Driven Lung Adenocarcinomas

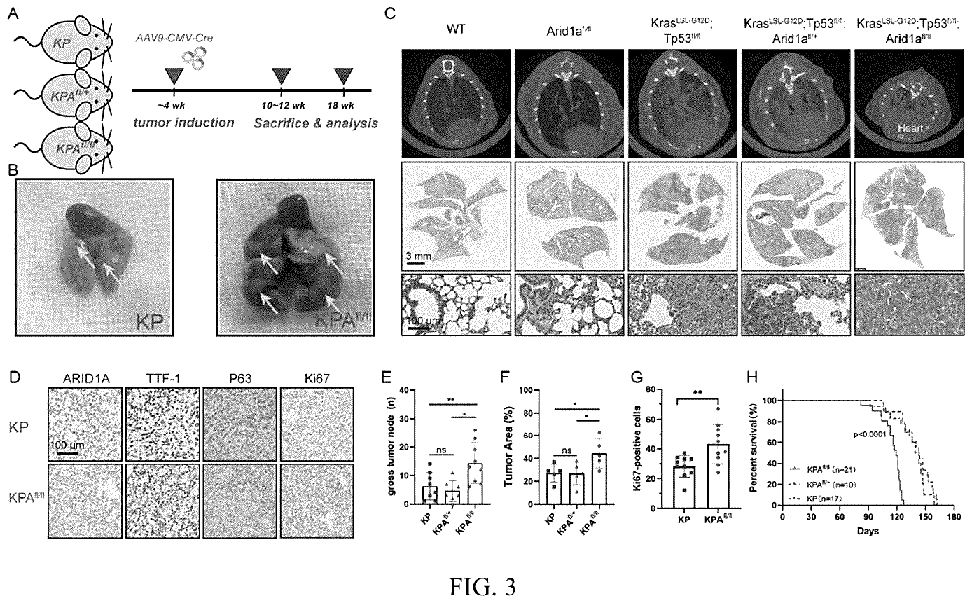

KrasLSL-G12 (C57BL/6-Krasem4 (LSL-G12D) Smoc, NM-KI-190003) and Tp53fl/fl (C57BL/6-Tp53tm2Smoc, NM-CKO-18005, denote as Tp53flox/flox mice) mouse strains were obtained from Shanghai Model Organisms Center, Inc. ARID1A (027717-STOCK Aridlatm1.1Zhwa/J, denote as ARID1Aflox/flox mice) mouse strains were obtained from Jackson Laboratory. KrasLSL-G12D, Tp53fl/fl (KP); KrasLSL-G12D, Tp53fl/fl, ARID1Afl/+ (KPAfl/+) and KrasLSL-G12D, Tp53fl/fl (KP); KrasLSL-G12D, Tp53fl/fl, ARID1Afl/fl (KPAfl/fl) mouse models were obtained by crossbreeding of the mouse strains. 4-week-old mice were delivered with the viruses via tracheal intubation, causing lung infections with type 9 adeno-associated viruses expressing the Cre recombinase (AAV9-CMV-Cre). Dissection and analysis were conducted at 10-12 weeks (the experimental protocol was shown in FIG. 3 panel A). Anatomical observation of the lungs of KP mice and KPAfl/fl mice suggested that ARID1A inactivation significantly promoted the tumorigenic ability of mouse lung tissues under the background of KrasG12D overexpression and Tp53 deletion (as shown in FIG. 3 panel B).

At 14 weeks after virus pulmonary perfusion, microCT was used to monitor the lung tumor burdens of wild-type (WT), KP, and KPAfl/+KPAfl/fl mice, and the lungs of the mice were dissected for HE staining analysis. Combining the results of microCT and HE staining analysis, it could be concluded that ARID1A heterozygous mutations did not induce the genesis of lung tumors. However, the complete deletion of ARID1A could greatly promote tumor progression in KP mice (as shown in FIG. 3 panel C).

Then, immunohistochemical analysis of lung tumor markers TTF-1, P63, ARID1A, and KI67 was performed on the lungs of KP and KPAfl/fl mice, and immunohistochemical staining confirmed a decrease in ARID1A protein levels in KPAfl/fl tumors. At the same time, TTF-1 (also known as Nkx2.1) was positive and p63 was negative in the nucleus, indicating that KPAfl/fl tumors had obvious lung adenocarcinoma features (as shown in FIG. 3 panels D and G).

The tumor area and number of visible tumor nodes on the lung surface of mice in KP, KPAfl/+, and KPA fl/fl groups were compared at 14 weeks: KPAfl/fl mice showed a significant increase in tumor number and tumor area compared with the KP group or KPAfl/+ group, while KP and KPAfl/+ mice showed a similar tumor number and tumor area. The above results indicated that the complete deletion of ARID1A could greatly promote the tumorigenesis of mouse lungs under the background of KrasG12D overexpression and Tp53 deletion, while ARID1A heterozygous mutations did not affect the tumorigenesis of Kras-driven tumor models (as shown in FIG. 3 panels E-F). Survival curves were plotted based on the survival time of lung tumor-burden mice in KP, KPAfl/+, and KPAfl/fl groups, and the overall survival rate of KPAfl/fl mice was lower (as shown in FIG. 3 panel H). The overall survival rate of KPAfl/fl mice was consistent with that of the mice phenotype with a higher lung tumor burden.

The above experimental results indicate that the ARID1A deletion promotes the genesis and development of lung tumors. Mice with ARID1A mutations have more malignant tumor phenotypes and higher tumor burdens, and overall survival rates are impaired.

### Example 3 Pathogenic Mechanism of ARID1A Deletion Promoting the Genesis and Development of In Vivo Lung Adenocarcinomas—Affecting Glycolysis Levels

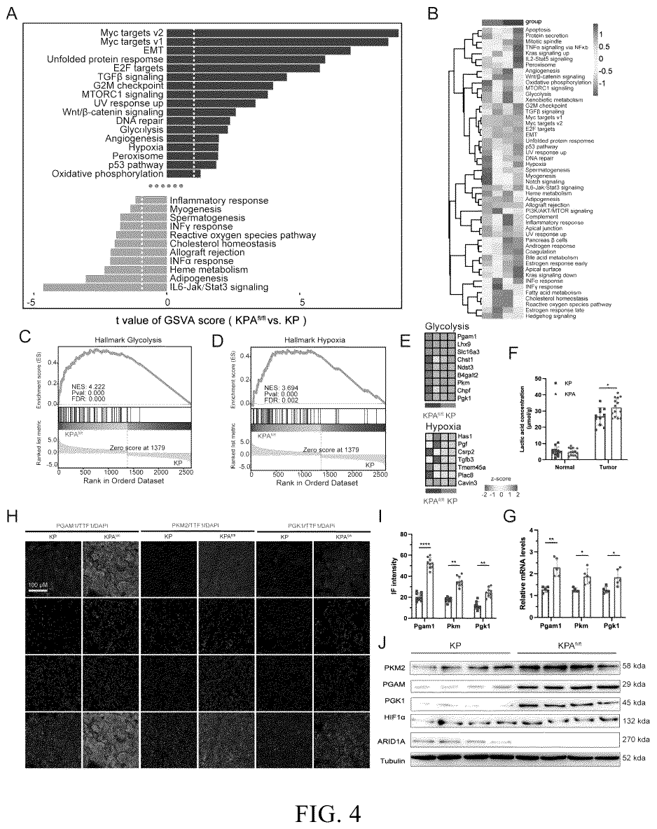

2931 genes with significant differential expression after ARID1A deletion were identified using RNA sequencing. These genes were involved in several pathways related to tumor progression, such as glycolysis, hypoxia, EMT, mTORC signaling, and Myc targets, etc. The results also showed that hypoxia and glycolysis signals were significantly upregulated in KPAfl/fl lung cancers (as shown in FIG. 4 panels A-D). Heat map analysis was performed on key genes involved in glycolysis and hypoxia pathways in KP and KPAfl/fl lung cancer tissues. It was found that Pgam1, Pkm, and Pgk1 encoding glycolysis promoting enzymes, as well as the key hypoxia pathway factor Hif-la, were upregulated in KPAfl/fl tumors (as shown in FIG. 4 panel E). Meanwhile, the concentration of lactic acid, a metabolite of glycolysis, also increased in the tumors of KPAfl/fl group (as shown in FIG. 4 panel F). Using q-PCR to analyze the expression levels of glycolysis genes in KP (n=9) and KPAfl/fl (n=11) tumors, the results showed that the expressions of glycolysis genes were significantly upregulated in KPAfl/fl tumors. Immunofluorescence and Western blot staining methods were further used and had confirmed that in the absence of ARID1A, the expressions of PGAM1, PKM2, and PGK1 proteins in tumor tissues were upregulated (FIG. 4 panels G-J). The expression level of hypoxia factor Hif-1α was not sensitive to the ARID1A deletion (FIG. 4 panel J).

The above experimental results indicate that the ARIDI deletion promotes the genesis and development of in vivo lung adenocarcinomas, and the mechanism thereof is to enhance glycolysis by upregulating Pgam1, Pkm, and Pgk1.

### Example 4 Pathogenic Mechanism of ARID1A Deletion Promoting the Genesis and Development of In Vivo Lung Adenocarcinomas—Remodeling the Chromatin State

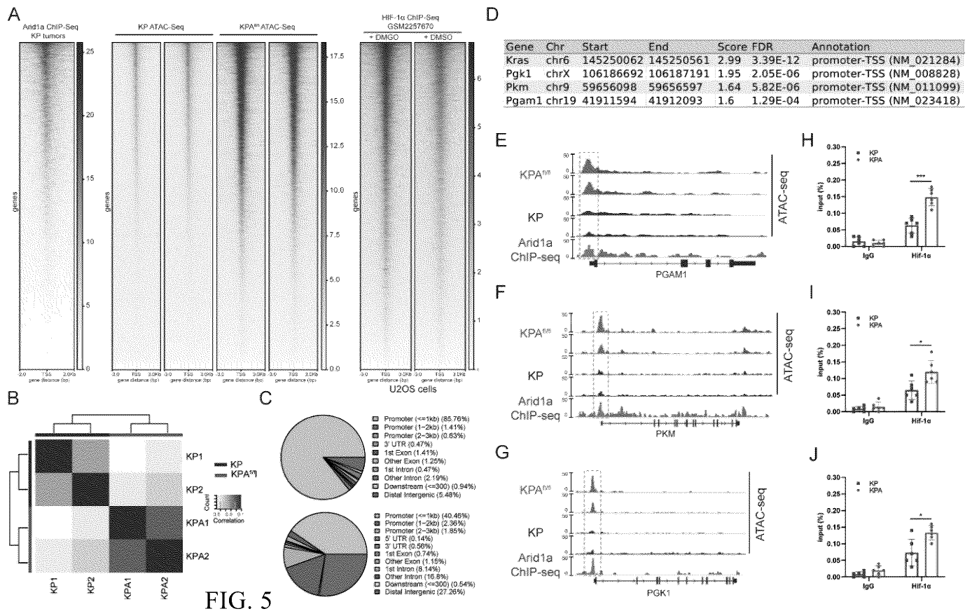

The content herein was also based on KP and KPAfl/fl induced lung cancer models. Firstly, based on the CHIP-seq method, the binding of ARID1A to genomic DNA in the context of KP tumors was analyzed. The results showed that ARID1A could bind to a large number of gene promoter regions (within a range of transcription initiation site #3.0 kb), and the promoter regions of the three genes, Pgam1, Pkm and Pgk1, also belonged to the ARID1A-binding sites (FIG. 5 panel A, left).

Secondly, the ATAC-seq method was used to analyze the chromosome state, and the research results showed that the ARID1A deletion (KPAfl/fl group) could enhance chromatin opening as a whole (FIG. 5 panels A, B), and most of the chromatin opening regions were concentrated at the transcription initiation site (FIG. 5 panel C, wherein Up referred to the upregulated peak, and Down referred to the downregulated peak). Diffbind analysis showed that the ARID1A deletion (KPAfl/fl group) significantly opened the transcription initiation regions of genes such as Pgam1, Pkm, and Pgk1 (FIG. 5 panel D). The open regions highly overlapped with the ARID1A-binding sites in the CHIP-seq experiment, indicating that the ARID1A deletion promoted chromatin accessibility in the promoter regions of genes such as Pgam1, Pkm, and Pgk1 in lung cancer cells.

Finally, the present study further correlated the interaction of ARID1A-hypoxia-glycolysis in lung cancers. Hypoxia was one of the typical microenvironmental characteristics of tumors, and early transcriptome sequencing results suggested that the ARID1A deletion could promote the activation of the tumor hypoxia signaling pathway FIG. 4 panels D, E). Through analyzing the public database (GSM2257670), it was found that Hif-1α could bind to the promoter regions of the three genes Pgam1, Pkm, and Pgk1.

Notably, the present invention confirmed that the ARID1A deletion (KPAfl/fl group) could significantly promote the binding of Hif-1α to promoter regions of genes such as Pgam1 (FIG. 5 panel H), Pkm (FIG. 5 panel I), and Pgk1 (FIG. 5 panel J).

The above results suggest that the ARID1A inactivation can promote chromatin opening in lung cancer cells, making it easier for Hif-la to bind to the promoter regions of the three genes Pgam1, Pkm and Pgk1, thereby increasing the expressions of Pgam1, Pkm and Pgk1, causing higher levels of glycolysis in tumor cells, and leading to intensified tumor cell progression.

The present invention provides a pathogenic mechanism of ARID1A deletion promoting the genesis and development of in vivo lung adenocarcinomas, which provides a series of new targets for the treatment of ARID1A-deficient lung cancers.

### Example 5 Glycolysis Inhibitors can Interfere with the Progression of ARID1A-Deficient Lung Cancers

In Example 4 of the present invention, the biological mechanism of ARID1A deletion promoting lung cancer progression by increasing glycolysis levels was described. In this example, one or more strategies were used to intervene in glycolysis levels, and whether the method(s) could inhibit the progression of ARID1A-deficient lung cancers was observed. In one embodiment, the present invention used a clinical drug 2-DG to inhibit glucose metabolism levels, including glycolysis levels of tumor cells. In another embodiment, the present invention used lentivirus-mediated CRISPR/Cas9 to interfere with the expression of Pgam1 gene (pSECC-sgPgaml group), wherein the nucleotide sequences of the sgRNA were as shown in SEQ ID NO: 4 and SEQ ID NO: 5. In another embodiment, the present invention used lentivirus-mediated CRISPR/Cas9 to interfere with the expression of Pkm gene (pSECC-sgPkm group), wherein the nucleotide sequences of the shRNA were as shown in SEQ ID NO: 6 and SEQ ID NO: 7.

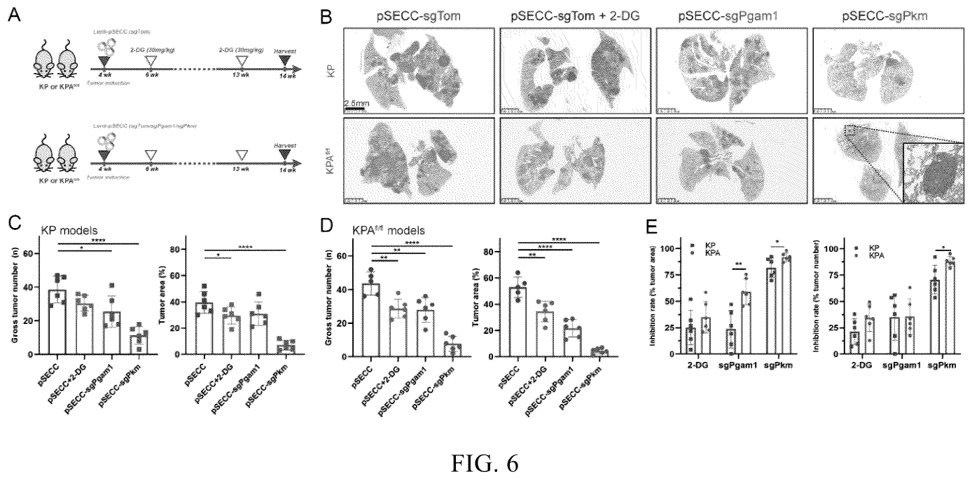

Firstly, 4-week-old KP and KPAfl/fl mouse systems were established. Secondly, AAV9-CMV-Cre virus induction was performed on mice. At the same time, lentivirus-mediated Cas9 and sgRNA were administered respectively to target a key gene Pgam1 or Pkm in glycolysis for intervention, with 2-DG drug treatment as another experimental group. SgTom was set as a negative control group, which did not target any endogenous gene. The experimental processing scheme was shown in FIG. 6 panel A. Finally, a multiplex detection method was used to evaluate the intervention effect of each treatment group.

Researches found that intervention with 2-DG, sgPgam1, and sgPkm could to some extent inhibit tumor progression caused by the ARID1A deletion. Among them, knocking out the Pkm gene could effectively reduce the in vivo tumor burden (FIG. 6 panels A-E). Therefore, intervention with Pgam1, Pkm or Pgk1, as well as other controllable methods to intervene in glycolysis, can serve as drug targets for the treatment of ARID1A-deficient lung cancers.

### Example 6

When BRD2 inhibition and ARID1A mutation coexist, it causes the death of ovarian cancer cells. Inhibition of BRD2 can reduce the expression levels of SWI/SWF complexes, and BETi small molecules can inhibit the BRD2 family protein BET. Therefore, BETi small molecules have specific inhibitory effects on ARID1A-deficient ovarian cancers. On this theoretical basis, the present invention investigated the inhibition of ARID1A-deficient lung cancers by BETi small molecules based on organoid models and in vivo animal models.

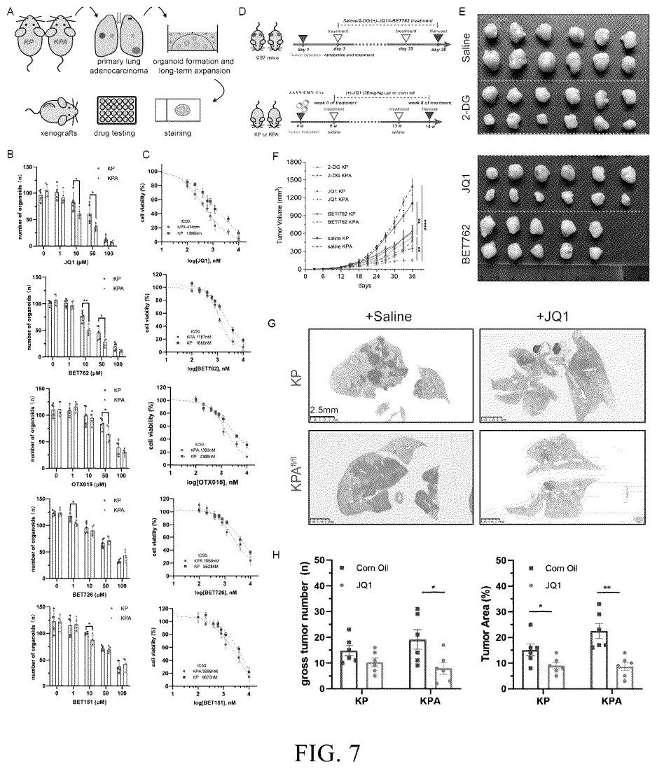

Firstly, lung tumor cells of KP and KPAfl/fl mice were taken for in vitro organoid culture and expansion, and drug sensitivity experiments of five widely used BET inhibitors were conducted on tumor-derived organoids (as shown in FIG. 7 panel A). To test the effects of drugs on the formation of tumor-derived organoids, we seeded the tumor-derived organoids into culture media containing different concentrations of JQ1, Bet762, OTX015, Bet726, and Bet151. On the 7th day, the number of organoids decreased with the increase of drug concentration, and various drugs exhibited different inhibitory effects on the formation of organoids. The results indicated that JQ1 and BET762 selectively impaired the formation of KPAfl/fl tumor-derived organoids at concentrations of 10 μm and 50 μm. JQ1 caused greater damage to the survival ability of ARID1A-deficient tumor organoids than ARID1A wild-type organoids (FIG. 7 panels B, C).

Secondly, tumor xenotransplantation was performed in mice, and KP and KPAfl/fl tumor organoids were xenotransplanted subcutaneously into C57 mice to test the effects of 2-DG, JQ1, and Bet762 on the growth of in vivo lung cancers (FIG. 7 panel D). During the experiment, tumor volumes were measured every 3 days. By comparing tumor volumes and weights, it was confirmed that tumors with ARID1A knockout were more sensitive to the treatment of JQ1 (FIG. 7 panels E, F).

Finally, the sensitivity of BETi small molecules to ARID1A-deficient cells was tested on transgenic model mice (KP and KPAfl/fl groups) (FIG. 7 panel D). The research results showed that by comparing the number and area of tumors, it was confirmed that ARID1A-deficient lung cancer cells were sensitive to JQ1 (FIG. 7 panels G, H).

The results of the above examples of the present invention indicate that the present invention provides efficacy data of BETi small molecule inhibitors on the ARID1A-deficient lung cancer.

All literatures mentioned in the present invention are incorporated herein by reference, as though each one is individually incorporated by reference. In addition, it should be understood that, after reading the above teachings of the present invention, those skilled in the art can make various changes or modifications to the present invention, and all these equivalents also fall within the scope of claims as defined in the appended claims of the present application.

1. Use of a glycolysis inhibitor in the manufacture of a composition or preparation, wherein the composition or preparation is used for: (a) preventing and/or treating lung cancer, wherein the lung cancer is an ARID1A-negative lung cancer; and/or (b) inhibiting lung cancer cells, wherein the lung cancer cells are ARID1A-negative lung cancer cells.

2. The use of claim 1, wherein the ARID1A-negative refers to a significant decrease in ARID1A expression and/or activity compared to normal control cells.

3. The use of claim 1, wherein the lung cancer is an ARID1A-deficient lung cancer.

4. The use of claim 1, wherein the lung cancer is selected from the group consisting of: adenocarcinoma, squamous cell carcinoma, and a combination thereof.

5. The use of claim 1, wherein the glycolysis inhibitor is selected from the group consisting of:
(Z1) a Hif-1α inhibitor;
(Z2) a Pgk1 inhibitor;
(Z3) a Pgam1 inhibitor;
(Z4) a Pkm2 inhibitor;
(Z5) any combination of Z1 to Z4 mentioned above.

6. The use of claim 1, wherein the glycolysis inhibitor is selected from the group consisting of: small molecule compound, antibody, antisense nucleic acid, gene editing drug, and a combination thereof.

7. The use of claim 1, wherein the glycolysis inhibitor is selected from the group consisting of: a BETi small molecule compound, a 2-DG small molecule, and a combination thereof.

8. Use of an ARID1A gene, mRNA, cDNA, protein, or a detection reagent thereof in the manufacture of a kit, wherein the kit is used for one or more uses selected from the group consisting of:
(i) evaluating whether a lung cancer patient is suitable for treatment with a glycolysis inhibitor; and/or
(ii) evaluating the prognosis of a lung cancer patient treated with a glycolysis inhibitor.

9. The use of claim 8, wherein the detection reagent comprises:
(a) an ARID1A-specific antibody, an ARID1A-specific binding molecule; and/or
(b) a primer or a primer pair, a probe, or a chip for specific amplification of ARID1A mRNA or ARID1A cDNA.

10. A product combination, wherein the product comprises:
(a) a detection reagent or a kit containing the detection reagent, wherein the detection reagent is a detection reagent for detecting ARID1A gene, mRNA, cDNA, protein, and a combination thereof; and
(b) a pharmaceutical composition, wherein the pharmaceutical composition comprises a glycolysis inhibitor as an active ingredient and a pharmaceutically acceptable carrier.


SEQUENCE LISTING (7 sequences)

SEQ ID NO: 1 - protein, 2285 aa, Homo sapiens
MAAQVAPAAASSLGNPPPPPPSELKKAEQQQREEAGGEAAAAAAAERGEMKAAAGQESEGPAVGPPQPLGKELQDGAESNGGGGGGGAGSGGGPGAEPDLKNSNGNAGPRPALNNNLTEPPGGGGGGSSDGVGAPPHSAAAALPPPAYGFGQPYGRSPSAVAAAAAAVFHQQHGGQQSPGLAALQSGGGGGLEPYAGPQQNSHDHGFPNHQYNSYYPNRSAYPPPAPAYALSSPRGGTPGSGAAAAAGSKPPPSSSASASSSSSSFAQQRFGAMGGGGPSAAGGGTPQPTATPTLNQLLTSPSSARGYQGYPGGDYSGGPQDGGAGKGPADMASQCWGAAAAAAAAAAASGGAQQRSHHAPMSPGSSGGGGQPLARTPQPSSPMDQMGKMRPQPYGGTNPYSQQQGPPSGPQQGHGYPGQPYGSQTPQRYPMTMQGRAQSAMGGLSYTQQIPPYGQQGPSGYGQQGQTPYYNQQSPHPQQQQPPYSQQPPSQTPHAQPSYQQQPQSQPPQLQSSQPPYSQQPSQPPHQQSPAPYPSQQSTTQQHPQSQPPYSQPQAQSPYQQQQPQQPAPSTLSQQAAYPQPQSQQSQQTAYSQQRFPPPQELSQDSFGSQASSAPSMTSSKGGQEDMNLSLQSRPSSLPDLSGSIDDLPMGTEGALSPGVSTSGISSSQGEQSNPAQSPFSPHTSPHLPGIRGPSPSPVGSPASVAQSRSGPLSPAAVPGNQMPPRPPSGQSDSIMHPSMNQSSIAQDRGYMQRNPQMPQYSSPQPGSALSPRQPSGGQIHTGMGSYQQNSMGSYGPQGGQYGPQGGYPRQPNYNALPNANYPSAGMAGGINPMGAGGQMHGQPGIPPYGTLPPGRMSHASMGNRPYGPNMANMPPQVGSGMCPPPGGMNRKTQETAVAMHVAANSIQNRPPGYPNMNQGGMMGTGPPYGQGINSMAGMINPQGPPYSMGGTMANNSAGMAASPEMMGLGDVKLTPATKMNNKADGTPKTESKSKKSSSSTTTNEKITKLYELGGEPERKMWVDRYLAFTEEKAMGMTNLPAVGRKPLDLYRLYVSVKEIGGLTQVNKNKKWRELATNLNVGTSSSAASSLKKQYIQCLYAFECKIERGEDPPPDIFAAADSKKSQPKIQPPSPAGSGSMQGPQTPQSTSSSMAEGGDLKPPTPASTPHSQIPPLPGMSRSNSVGIQDAFNDGSDSTFQKRNSMTPNPGYQPSMNTSDMMGRMSYEPNKDPYGSMRKAPGSDPFMSSGQGPNGGMGDPYSRAAGPGLGNVAMGPRQHYPYGGPYDRVRTEPGIGPEGNMSTGAPQPNLMPSNPDSGMYSPSRYPPQQQQQQQQRHDSYGNQFSTQGTPSGSPFPSQQTTMYQQQQQNYKRPMDGTYGPPAKRHEGEMYSVPYSTGQGQPQQQQLPPAQPQPASQQQAAQPSPQQDVYNQYGNAYPATATAATERRPAGGPQNQFPFQFGRDRVSAPPGTNAQQNMPPQMMGGPIQASAEVAQQGTMWQGRNDMTYNYANRQSTGSAPQGPAYHGVNRTDEMLHTDQRANHEGSWPSHGTRQPPYGPSAPVPPMTRPPPSNYQPPPSMQNHIPQVSSPAPLPRPMENRTSPSKSPFLHSGMKMQKAGPPVPASHIAPAPVQPPMIRRDITFPPGSVEATQPVLKQRRRLTMKDIGTPEAWRVMMSLKSGLLAESTWALDTINILLYDDNSIMTFNLSQLPGLLELLVEYFRRCLIEIFGILKEYEVGDPGQRTLLDPGRFSKVSSPAPMEGGEEEEELLGPKLEEEEEEEVVENDEEIAFSGKDKPASENSEEKLISKFDKLPVKIVQKNDPFVVDCSDKLGRVQEFDSGLLHWRIGGGDTTEHIQTHFESKTELLPSRPHAPCPPAPRKHVTTAEGTPGTTDQEGPPPDGPPEKRITATMDDMLSTRSSTLTEDGAKSSEAIKESSKFPFGISPAQSHRNIKILEDEPHSKDETPLCTLLDWQDSLAKRCVCVSNTIRSLSFVPGNDFEMSKHPGLLLILGKLILLHHKHPERKQAPLTYEKEEEQDQGVSCNKVEWWWDCLEMLRENTLVTLANISGQLDLSPYPESICLPVLDGLLHWAVCPSAEAQDPFSTLGPNAVLSPQRLVLETLSKLSIQDNNVDLILATPPFSRLEKLYSTMVRFLSDRKNPVCREMAVVLLANLAQGDSLAARAIAVQKGSIGNLLGFLEDSLAATQFQQSQASLLHMQNPPFEPTSVDMMRRAARALLALAKVDENHSEFTLYESRLLDISVSPLMNSLVSQVICDVLFLIGQS

SEQ ID NO: 2 - protein, 2283 aa, Mus musculus
MAAQVAPAAASSLGNPPPPPSELKKAEQQQREEAGGEAAAAAAERGEMKAAAGQESEGPAVGPPQPLGKELQDGAESNGGGGGGGAGSGGGPGAEPDLKNSNGNAGPRPALNNNLPEPPGGGGGGGSSSSDGVGAPPHSAAAALPPPAYGFGQAYGRSPSAVAAAAAAVFHQQHGGQQSPGLAALQSGGGGGLEPYAGPQQNSHDHGFPNHQYNSYYPNRSAYPPPPQAYALSSPRGGTPGSGAAAAAGSKPPPSSSASASSSSSSFAQQRFGAMGGGGPSAAGGGTPQPTATPTLNQLLTSPSSARGYQGYPGGDYGGGPQDGGAGKGPADMASQCWGAAAAAAAAAAAVSGGAQQRSHHAPMSPGSSGGGGQPLARTPQSSSPMDQMGKMRPQPYGGTNPYSQQQGPPSGPQQGHGYPGQPYGSQTPQRYPMTMQGRAQSAMGSLSYAQQIPPYGQQGPSAYGQQGQTPYYNQQSPHPQQQPPYAQQPPSQTPHAQPSYQQQPQTQQPQLQSSQPPYSQQPSQPPHQQSPTPYPSQQSTTQQHPQSQPPYSQPQAQSPYQQQQPQQPASSSLSQQAAYPQPQPQQSQQTAYSQQRFPPPQELSQDSFGSQASSAPSMTSSKGGQEDMNLSLQSRPSSLPDLSGSIDDLPMGTEGALSPGVSTSGISSSQGEQSNPAQSPFSPHTSPHLPGIRGPSPSPVGSPASVAQSRSGPLSPAAVPGNQMPPRPPSGQSDSIMHPSMNQSSIAQDRGYMQRNPQMPQYTSPQPGSALSPRQPSGGQMHSGVGSYQQNSMGSYGPQGSQYGPQGGYPRQPNYNALPNANYPNAGMAGSMNPMGAGGQMHGQPGIPPYGTLPPGRMAHASMGNRPYGPNMANMPPQVGSGMCPPPGGMNRKTQESAVAMHVAANSIQNRPPGYPNMNQGGMMGTGPPYGQGINSMAGMINPQGPPYPMGGTMANNSAGMAASPEMMGLGDVKLTPATKMNNKADGTPKTESKSKKSSSSTTTNEKITKLYELGGEPERKMWVDRYLAFTEEKAMGMTNLPAVGRKPLDLYRLYVSVKEIGGLTQVNKNKKWRELATNLNVGTSSSAASSLKKQYIQCLYAFECKIERGEDPPPDIFAAADSKKSQPKIQPPSPAGSGSMQGPQTPQSTSSSMAEGGDLKPPTPASTPHSQIPPLPGMSRSNSVGIQDAFPDGSDPTFQKRNSMTPNPGYQPSMNTSDMMGRMSYEPNKDPYGSMRKAPGSDPFMSSGQGPNGGMGDPYSRAAGPGLGSVAMGPRQHYPYGGPYDRVRTEPGIGPEGNMGTGAPQPNLMPSTPDSGMYSPSRYPPQQQQQQQQQHDSYGNQFSTQGTPSSSPFPSQQTTMYQQQQQNYKRPMDGTYGPPAKRHEGEMYSVPYSAGQGQPQQQQLPAAQSQPASQPQAAQPSPQQDVYNQYSNAYPASATAATDRRPAGGPQNQFPFQFGRDRVSAPPGSSAQQNMPPQMMGGPIQASAEVAQQGTMWQGRNDMTYNYANRQNTGSATQGPAYHGVNRTDEMLHTDQRANHEGPWPSHGTRQPPYGPSAPVPPMTRPPPSNYQPPPSMPNHIPQVSSPAPLPRPMENRTSPSKSPFLHSGMKMQKAGPPVPASHIAPTPVQPPMIRRDITFPPGSVEATQPVLKQRRRLTMKDIGTPEAWRVMMSLKSGLLAESTWALDTINILLYDDNSIMTFNLSQLPGLLELLVEYFRRCLIEIFGILKEYEVGDPGQRTLLDPGRFTKVYSPAHTEEEEEEHLDPKLEEEEEEGVGNDEEMAFLGKDKPSSENNEEKLVSKFDKLPVKIVQRNDPFVVDCSDKLGRVQEFDSGLLHWRIGGGDTTEHIQTHFESKIELLPSRPYVPCPTPPRKHLTTVEGTPGTTEQEGPPPDGLPEKRITATMDDMLSTRSSTLTDEGAKSAEATKESSKFPFGISPAQSHRNIKILEDEPHSKDETPLCTLLDWQDSLAKRCVCVSNTIRSLSFVPGNDFEMSKHPGLLLILGKLILLHHKHPERKQAPLTYEKEEEQDQGVSCDKVEWWWDCLEMLRENTLVTLANISGQLDLSPYPESICLPVLDGLLHWAVCPSAEAQDPFSTLGPNAVLSPQRLVLETLSKLSIQDNNVDLILATPPFSRLEKLYSTMVRFLSDRKNPVCREMAVVLLANLAQGDSLAARAIAVQKGSIGNLLGFLEDSLAATQFQQSQASLLHMQNPPFEPTSVDMMRRAARALLALAKVDENHSEFTLYESRLLDISVSPLMNSLVSQVICDVLFLIGQS

SEQ ID NO: 3 - DNA, 1026 bp, synthetic construct
  source 1..1026: mol_type = other DNA; note = Gene fragment containing Cre recombinase
tccaatctcctgactgttcaccagaacctccctgcgctgccagtagatgccactagcgatgaggtcaggaaaaatctcatggatatgtttagggatagacaggcgttttctgaacacacctggaaaatgctgcttagcgtgtgccgatcctgggcagcctggtgtaagctgaacaatcgcaaatggttccccgccgagccggaggacgtgcgcgattacctgctgtatctccaggcaagagggctggctgtcaagactatccagcagcacttgggccaactgaatatgctgcatcgacgcagcgggctcccccggcctagcgattcaaacgcagtctcccttgttatgaggagaattagaaaggaaaacgtagatgcgggtgagagggctaagcaggctctcgcttttgagcggactgatttcgaccaggtcagatccctgatggagaacagcgatcggtgccaggacatcaggaacctcgcatttctgggaattgcatataacacacttctgcgcatagctgagatcgcccggatcagagtgaaagacatcagtcgaacggacggcggccggatgcttattcatattggacgcacaaagacattggtcagcaccgctggcgttgaaaaggccttgtccctgggcgtaacgaagctggtggaaagatggatctcagtgtccggcgtggctgacgaccctaataattacttgttctgtcgagtgagaaaaaacggagtcgccgcgccctctgccaccagccaattgagtacacgggcccttgaagggatctttgaggcaacccaccgactcatatacggagccaaggatgacagtggccagaggtatctcgcctggtcaggtcattctgctagggtgggggccgcacgagacatggcgcgggcaggagtctccataccagagattatgcaagctggaggttggacaaatgtgaacatcgttatgaactatatccgcaatcttgactctgaaaccggggccatggtgagactgctcgaagatggtgac

SEQ ID NO: 4 - DNA, 21 bp, synthetic construct
  source 1..21: mol_type = other DNA; note = Nucleotide sequence of a Pgam1 inhibitor-sgRNA
ggattgctctcttctgcacag

SEQ ID NO: 5 - DNA, 21 bp, synthetic construct
  source 1..21: mol_type = other DNA; note = Nucleotide sequence of a Pgam1 inhibitor-sgRNA
ttgaccagatgtggttgccag

SEQ ID NO: 6 - DNA, 21 bp, synthetic construct
  source 1..21: mol_type = other DNA; note = Nucleotide sequence of a Pkm2 inhibitor-shRNA
ggggcagagtcaatgtccagg

SEQ ID NO: 7 - DNA, 21 bp, synthetic construct
  source 1..21: mol_type = other DNA; note = Nucleotide sequence of a Pkm2 inhibitor-shRNA
ccgaagccacacagtgaagca